(19) United States
(12) Patent Application Publication
(10) Pub. No.: US 2025/0345525 A1
(43) Pub. Date: Nov. 13, 2025

(54) MULTI-LAYERED SYRINGE WITH A THREADED CONNECTION PART AND METHOD OF MANUFACTURING SAME

(71) Applicant: Pfizer Inc., New York, NY (US)

(72) Inventors: Daniel Mazur, Libertyville, IL (US); Charles Daniel Menolascino, Algonquin, IL (US); Benjamin L. Rush, Glenview, IL (US); John Glen Sundquist, Glen Ellyn, IL (US); Ming Zhou, Lake Forest, IL (US)

(73) Assignee: Pfizer Inc., New York, NY (US)

(21) Appl. No.: 18/860,320

(22) PCT Filed: Apr. 21, 2023
(86) PCT No.: PCT/IB2023/054121

§ 371 Date: Oct. 25, 2024

Related U.S. Application Data
(60) Provisional application No. 63/334,963, filed on Apr. 26, 2022.

(51) Int. Cl. A61M 5/31; A61L 29/04; A61L 29/06; A61M 5/34; B29C 45/16; B29K 23/00; B29K 27/18; B29K 69/00; B29K 77/00; B29K 105/00; B29L 31/00

(52) U.S. Cl. CPC A61M 5/3129; A61L 29/041; A61L 29/06; A61M 5/347; A61M 2207/10; B29C 45/16; B29K 2023/086; B29K 2027/18; B29K 2069/00; B29K 2077/00; B29K 2105/0044; B29L 2031/7544

(57) ABSTRACT

The invention relates to a syringe comprising an elongated main body having a nozzle formed at a distal end thereof and a threaded connection part at least partially surrounding the nozzle. The main body is made by co-injection molding from at least two different materials and the threaded connection part is made of a plastic material by injection molding, with the main body and the threaded connection part being overmolded one to the other. The invention also relates to a method of manufacturing such a syringe.

### TECHNICAL FIELD

The present invention relates to syringes, in particular for containing pharmaceutical products for administration to human subjects, and to methods for the manufacturing of such syringes.

More specifically the invention relates to a syringe comprising an elongated main body extending along an axis and comprising a nozzle formed at a distal end thereof, the main body having a multi-layered structure over at least a part of its length and being made by co-injection molding from at least two different materials.

### BACKGROUND OF THE INVENTION

In the pharmaceutical field, synthetic materials such as polypropylene have been long used for manufacturing syringe main bodies as an alternative to making them from glass. For prefilled syringes containing medicaments particularly sensitive to gas (in particular oxygen), moisture or U.V. light ingress that may affect the product stability over time, it has been proposed to provide the main body with an additional layer made of a different material providing the required barrier function.

It is known that such syringe main bodies can advantageously be molded by a co-injection process, wherein two different materials are injected into the same mold for forming a sandwich structure having an inner and an outer layers made of the first material, e.g. polypropylene, and a core layer extending between the inner and outer layers. The core layer is made of the second material, e.g. ethylene-vinyl alcohol copolymer (EVOH), that provides the barrier function.

It may be desirable to produce syringes of this type that also include a male threaded connection part, typically referred to as Luer thread tip, for connection of the syringe to an IV line or a needle.

However, available co-injection molding technologies cannot be used to mold a Luer thread tip integrally with the syringe body, due to a challenge to form a consistent multi-layer structure in the Luer thread.

Other options may be available to produce syringes with a threaded connection that would involve a step of separately molding the threaded connection part and a step of attaching the same to the co-injected body. For example, this could be achieved by an ultrasonic or glue welding step.

There are multiple downsides associated with such methods, in particular the manufacturing complexity, cycle time and cost. Also, the bonding step for attaching the Luer connection piece to the main body may generate particulates, which is highly undesirable for an application to medication-prefilled plastic syringe.

It is therefore an objective of the invention to provide a syringe of the aforementioned type and an associated method of manufacturing that involve a reduced manufacturing complexity, cycle time, cost, and particulate generation.

### SUMMARY OF THE INVENTION

According to a first aspect of the present invention, it is provided a syringe comprising an elongated main body extending along an axis and comprising a nozzle formed at a distal end thereof, the main body having a multi-layered structure over at least a part of its length and being made by co-injection molding from at least two different materials, wherein the syringe further comprises a threaded connection part at least partially surrounding the nozzle, the threaded connection part being made of a plastic material by injection molding, and wherein the main body and the threaded connection part are overmolded one to the other.

According to preferred embodiments of the invention:
the main body includes an outer layer and an inner layer, both made of a first material, and a core layer sandwiched between the outer and inner layers, the core layer being made of a second material and providing a gas and/or moisture barrier;
the first material includes a polyolefin resin;
the polyolefin resin includes a polypropylene, a cycloolefin polymer (COP), or a cycloolefin copolymer (COC);
the first material includes a polyester resin;
the polyester resin includes a polyethylene terephthalate (PET), a polyethylene terephthalate glycol (PETG), or a polycyclohexylenedimethylene terephthalate glycol (PCTG);
the first material includes a polycarbonate resin; and

-the first material includes a blend of a polycarbonate resin and a polyester resin.

In a still preferred embodiment, the second material may include ethylene-vinyl alcohol copolymer (EVOH).

Alternatively, the second material may include a polyamide resin.

Alternatively, the second material may include a polychlorotrifluoroethylene (PCTFE) resin.

Preferred embodiments may include one or several of the following features:
the second material includes an additive comprising a U.V. stabilizer, an oxygen scavenger, or a mixture thereof;
the threaded connection part is made of a third material that is either identical to the first material or a blend of the first material and a reinforcing additive, so as to achieve a suitable connection between the threaded connection part and the main body;
the main body comprises a cylindrical barrel and a frustoconical portion connecting a distal end of the cylindrical barrel to the nozzle, and wherein the threaded connection part is bonded to the main body at an external surface of the frustoconical portion;
the frustoconical portion has at least one lug outwardly protruding from its external surface for enhancing the bonding with the threaded connection part;

-the frustoconical portion has a plurality of outwardly protruding lugs regularly arranged about the axis.

In a further aspect, it is provided a method of manufacturing a syringe according to the invention, wherein the threaded connection part is formed by a first injection molding shot and the main body is overmolded to the threaded connection part by a second co-injection molding shot.

Alternatively, the main body is formed by a first co-injection molding shot and the threaded connection part is overmolded to the main body by a second injection molding shot.

Preferably, the first and second molding shots are sequentially carried out in a single mold.

### BRIEF DESCRIPTION OF THE DRAWINGS

A preferred embodiment of the invention will now be described in more details, with reference to the following drawings wherein:

FIG. 1 is a perspective view of a syringe according to a preferred embodiment of the invention;

FIG. 2 is a cross-sectional view of the syringe of FIG. 1 in an axial plane comprising the axis X shown on FIG. 1;

FIG. 3 is a perspective view of the main body alone of the syringe of FIGS. 1 and 2;

FIG. 4 is a cross-sectional view in the axial plane of the main body represented on FIG. 3;

FIG. 5 is a cross-sectional view in the axial plane of the main body and the threaded connection part of the syringe of FIGS. 1 and 2;

FIG. 6 is an enlarged view of detail D shown on FIG. 5.

### DETAILED DESCRIPTION OF A PREFERRED EMBODIMENT

### Definitions

The following definitions will be used in the present description and claims:
the term “co-injection” refers to methods of injection molding wherein two or more different materials are injected in the same mold so as to form a molded piece made of at least two different materials;
the term “distal” refers to a location, or direction, that is close to, or oriented toward, the outlet port for the content of the syringe; and
the term “proximal” refers to a location, or direction, that is close to, or oriented toward, the prehension part of the syringe.

The invention will now be further illustrated by the following preferred embodiment illustrated on the Figures.

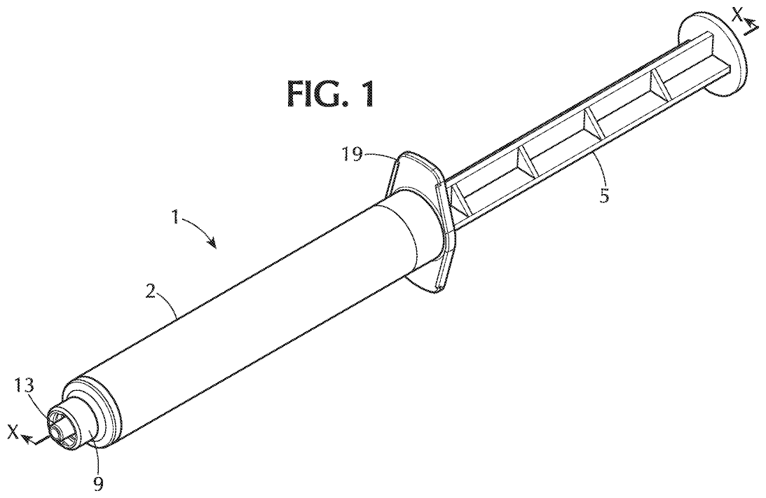

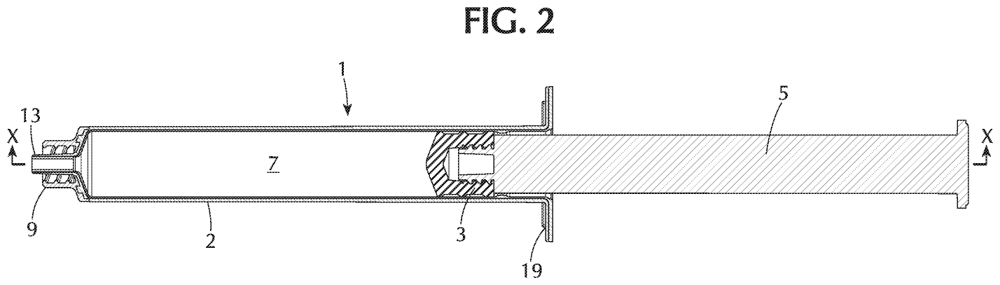

With reference to FIGS. 1 and 2, the syringe 1 comprises a main body 2 that is elongated and extends along an axis X. The syringe 1 further comprises a piston 3, typically made of an elastomeric material and arranged within the main body 2 to be axially displaced, and a piston rod 5 attached to the piston 3 and axially projecting from a proximal open end of the main body 2. The piston rod 3 is provided to be actuated by a user for displacing the piston 3. The main body 2 and the piston 3 define an inner volume 7 for containing a liquid product, in particular a pharmaceutical product to be administered to a patient. The syringe 1 further comprises, at a distal end thereof, a male threaded connection part 9 for connecting the syringe 1 to an IV line through a secure male-to-female connection, such as a Luer-Lock® connector, or to a needle.

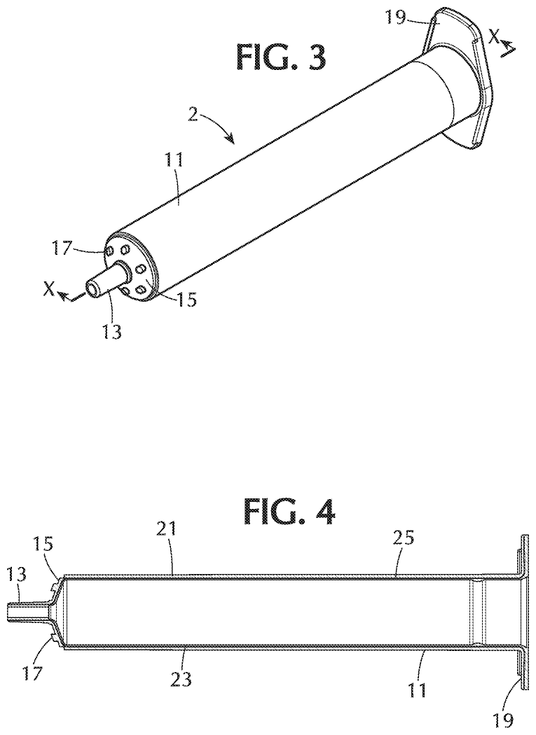

As better seen on FIGS. 3 and 4, showing the main body 2 alone i.e. the syringe 1 deprived of the piston 3, the piston rod 5 and the threaded connection part 9, the main body 2 includes a cylindrical barrel 11 slidably receiving the piston 3.

At its distal end, the main body 2 comprises a nozzle 13, in the form of a tapered tip, in fluid communication with the inner volume 7 for providing an outlet port for the liquid product contained in the syringe. The main body 2 further comprises a frustoconical portion 15 formed by a wall connecting a distal end of the cylindrical barrel 11 to the nozzle 13. The frustoconical portion 15 has on its external surface, and outwardly protruding therefrom, a plurality of lugs 17 regularly arranged about the axis X. The lugs 17 are provided for enhancing the bonding with the threaded connection part 9, as will be seen in the foregoing. Six such lugs 17 are provided in the illustrated embodiment.

At its proximal end, the main body 2 comprises a radially extending flange 19 forming a finger grip for a user.

It will be apparent from FIGS. 4-6 that the main body has a multi-layered structure. In this embodiment, the main body 2 is formed by a wall composed of three layers:
an outer layer 21 defining the external surface of the main body 2, with the lugs 17 of the frustoconical portion 15 being formed on said outer layer 21;
an inner layer 23 defining the internal surface of the main body 2 and intended to be in contact with the product and the piston 3; and
a core layer 25 sandwiched between the outer layer 21 and the inner layer 22.

In the preferred embodiment, the outer 21 and inner 23 layers are made of the same first material and the core layer 25 is made of a second material, different from the first material. It is however conceivable that the outer 21 and inner 23 layers may be made of different materials. As represented on the Figures, in the preferred embodiment, the main body 2 is comprised of three layers but it can be envisaged that it may be comprised of more than three layers, still made from at least two different materials.

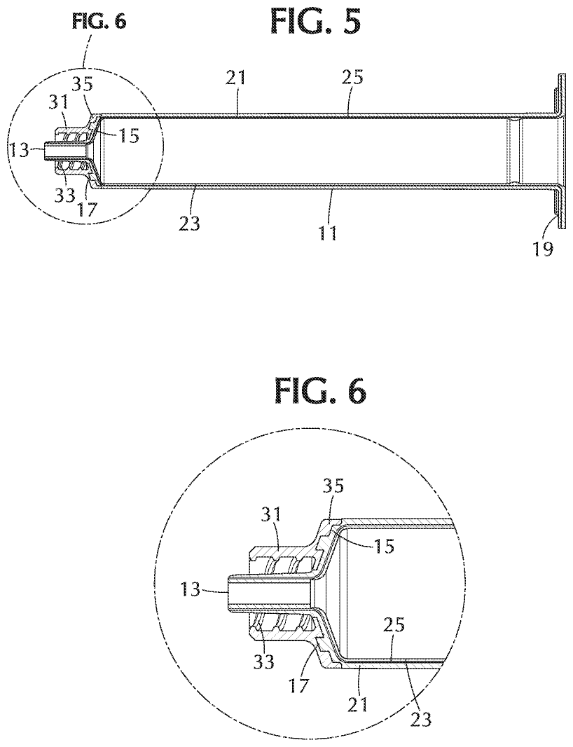

As can be seen on FIGS. 4-5, the multi-layer structure of the main body 2 extends over the whole length thereof i.e. from the distal end to the proximal end. Alternatively, such a multi-layer structure may be limited to a part of the length, in particular the part delimiting the inner volume 7 containing the product.

The main body 2, including the cylindrical barrel 11, the nozzle 13, the frustoconical portion 15 and the finger grip 19, is integrally made by a co-injection molding step from the first and second materials.

The first material is selected for certain properties required to constitute the outer 21 and inner 23 layers, such as the rigidity, the compatibility with the product to be contained in the syringe, the slidability, capability for sterilization, transparency, manufacturability, etc . . .

For example, the first material may include a polyolefin resin, such as a polyolefin resin including a polypropylene, a cycloolefin polymer (COP), or a cycloolefin copolymer (COC).

Particular examples of suitable polyolefin resins for use as the first material are listed below:
polypropylene homopolymer, for example Achieve™ Advanced U.S. Plant Pat. No. 1,605, Achieve™ Advanced U.S. Plant Pat. No. 6,282NE2 from ExxonMobil, or Bormed™ HD810MO from Borealis AG;
polypropylene random copolymer, for example ExxonMobil™ U.S. Plant Pat. No. 9,074MED, ExxonMobil™ U.S. Plant Pat. No. 9,102 from ExxonMobil, or Bormed™ RF830MO from Borealis AG;
a blend of polypropylene homopolymer and random copolymer, for example a blend of Achieve™ Advanced U.S. Plant Pat. No. 1,605 and ExxonMobil™ U.S. Plant Pat. No. 9,102 at a blend ratio between 10/90 to 90/10;
COP, a polymer produced by ring-opening polymerization of norbornene and followed by hydrogenation. COP, for example ZEONEX® 5000, ZEONEX® 690R, ZEONEX® 790R and ZEONOR® 1020R, is commercially available from ZEON Corporation;
COC, a copolymer typically produced by a ring-opening copolymerization of norborene and ethylene. COC is commercially available from Polyplastics USA, Inc., as TOPAS 8007, TOPAS 5013, TOPAS 6013 and TOPAS 6015.

Alternatively, the first material may include a polyester resin, such as a polyester resin including polyethylene terephthalate (PET), polyethylene terephthalate glycol (PETG), or poly cyclohexylenedimethylene terephthalate glycol (PCTG).

Particular examples of suitable polyester resins for use as the first material are listed below:
PET that is produced from condensation polymerization of ethylene glycol and dimethyl terephthalate (DMT) or terephthalic acid, for example Eastar™ EN058 from Eastman Chemicals;
PETG, glycol modified PET, that is produced by introducing glycol monomers into PET to improve material's strength, durability, impact resistance and high temperature compatibility, for example Eastar™ MN021, Eastar™ DN004 from Eastman Chemicals;
PCTG, a glycol-modified poly cyclohexylenedimethylene terephthalate, for example Tritan™ MX731, Tritan™ MX711 and Tritan™ SC830 from Eastman Chemicals.

Alternatively, the first material may include a polycarbonate resin.

Particular examples of suitable polyester resins for use as the first material are Makrolon® 2458, Makrolon® Rx2530, Makrolon® Rx1805, Apec® 1745 commercially available from Covestro; Lexan™ HP1R, HPS2R, and HPS7 commercially available from Sabic.

Alternatively, the first material includes a blend of a polycarbonate resin and a polyester resin.

Particular examples of suitable blends of polycarbonate and polyester resins for use as the first material are Makroblend® EC150, EL700, EL703 commercially available from Covestro; Xylex™ HX8300HP, HX7409HP, HX7509HP commercially available from Sabic.

The second material is selected for certain properties such as providing gas barrier, moisture barrier and/or U.V. light barrier.

For example, the second material may include ethylene-vinyl alcohol copolymer (EVOH), such as EVAL™ XEP-1248, EVAL™ XEP-1314, EVAL™ XEP-1283 and EVAL™ XEP-1303 from Kuraray Co., Ltd.

Alternatively, the second material may include a polyamide resin, such as Nylon-MXD6 MX nylon S6011 produced from m-xylenediamine (MXDA) by Mitsubishi Gas Chemical Co., or Nylon 6 UBE Nylon 1022B from UBE Industries, Ltd, or Selar® PA3426R from DuPont™.

Alternatively, the second material may include a polychlorotrifluoroethylene (PCTFE) resin, such as Aclar® Edge from Honeywell International Inc.

Optionally, the second material includes an additive comprising a U.V. stabilizer, an oxygen scavenger, or a mixture thereof.

With particular reference to FIGS. 5 and 6, it will be seen that the threaded connection part 9 comprises an axially extending sleeve 31 that coaxially surrounds the nozzle 13 over a part of the length thereof, whereby the distal end of the nozzle 13 projects from the sleeve 31 and is exposed. The sleeve 31 is provided with an inner thread 33. The threaded connection part 9 further comprises a base 35, by which the threaded connection part 9 is bonded to the external surface of the frustoconical portion 15. It will be noted that the lugs 17 project into the material forming the base 35, thus enhancing the bonding of the threaded connection part 9 to the main body 2.

In the represented embodiment, the threaded connection part 9 does not extend over the cylindrical barrel 11 and is only bonded to the main body 2 at a distal part thereof, specifically to the frustoconical portion 15 connecting the cylindrical barrel 11 to the nozzle 13.

In alternative embodiments (not illustrated), the threaded connection part 9 may further extend over a part or the entirety of the main body 2 and thus further be bonded to the cylindrical barrel 11.

The threaded connection part 9 is made in one piece by injection molding, preferably from a single plastic material.

The main body 2 and the threaded connection part 9 are made in a single mold, with the main body 2 and the threaded connection part 9 being overmolded one to the other.

Preferably, the threaded connection part 9 is formed by a first injection molding shot, from a single material, and the main body 2 is overmolded to the threaded connection part 9 by a second co-injection molding shot, from the first and second materials.

Alternatively, the order of the molding steps may be inverted, with the main body 2 formed by a first co-injection molding shot and the threaded connection part 9 is overmolded to the main body by a second injection molding shot.

The first and second molding shots, whether the co-injection molding shot is carried out first or second, are sequentially carried out in the single mold.

The single polymer or plastic material for forming the threaded connection part 9 may be a third material that is either identical to the first material or a blend of the first material and a reinforcing additive, so as to achieve a suitable connection between the threaded connection part 9 and the main body 2.

It will be appreciated that the two-shot overmolding method of the invention avoids a need of additional steps to assemble a Luer connection component onto a syringe body.

The invention in particular allows to avoid such any secondary welding step, thus reducing manufacturing complexity and cost. Furthermore, the invention avoids risks of particulate generation during the manufacturing process, that may cause contamination of the product contained in the syringe.

1. Syringe comprising an elongated main body (2) extending along an axis (X) and comprising a nozzle (13) formed at a distal end thereof, the main body (2) having a multi-layered structure over at least a part of its length and being made by co-injection molding from at least two different materials,
wherein the syringe (1) further comprises a threaded connection part (9) at least partially surrounding the nozzle (13), the threaded connection part (9) being made of a plastic material by injection molding,
and wherein the main body (2) and the threaded connection part (9) are overmolded one to the other.

2. Syringe according to claim 1, wherein the main body (2) includes an outer layer (21) and an inner layer (23), both made of a first material, and a core layer (25) sandwiched between the outer and inner layers, the core layer (25) being made of a second material and providing a gas and/or moisture barrier.

3. Syringe according to claim 2, wherein the first material includes a polyolefin resin.

4. Syringe according to claim 3, wherein the polyolefin resin includes a polypropylene, a cycloolefin polymer (COP), or a cycloolefin copolymer (COC).

5. Syringe according to claim 2, wherein the first material includes a polyester resin.

6. Syringe according to claim 5, wherein the polyester resin includes a polyethylene terephthalate (PET), a polyethylene terephthalate glycol (PETG), or a polycyclohexylenedimethylene terephthalate glycol (PCTG).

7. Syringe according to claim 2, wherein the first material includes a polycarbonate resin.

8. Syringe according to claim 7, wherein the first material includes a blend of a polycarbonate resin and a polyester resin.

9. Syringe according to claim 2, wherein the second material includes ethylene-vinyl alcohol copolymer (EVOH).

10. Syringe according to claim 2, wherein the second material includes a polyamide resin.

11. Syringe according to claim 2, wherein the second material includes a polychlorotrifluoroethylene (PCTFE) resin.

12. Syringe according to claim 2, wherein the second material includes an additive comprising a U.V. stabilizer, an oxygen scavenger, or a mixture thereof.

13. Syringe according to claim 2, wherein the threaded connection part (9) is made of a third material that is either identical to the first material or a blend of the first material and a reinforcing additive, so as to achieve a suitable connection between the threaded connection part (9) and the main body (2).

14. Syringe according to claim 1, wherein the main body (2) comprises a cylindrical barrel (11) and a frustoconical portion (15) connecting a distal end of the cylindrical barrel (11) to the nozzle (13), and wherein the threaded connection part (9) is bonded to the main body (2) at an external surface of the frustoconical portion (15).

15. Syringe according to claim 14, wherein the frustoconical portion (15) has at least one lug (17) outwardly protruding from its external surface for enhancing the bonding with the threaded connection part (9).

16. Syringe according to claim 15, wherein the frustoconical portion (15) has a plurality of outwardly protruding lugs (17) regularly arranged ab,out the axis.

17. Method of manufacturing a syringe according to claim 1 wherein the threaded connection part (9) is formed by a first injection molding shot and the main body (2) is overmolded to the threaded connection part (9) by a second co-injection molding shot.

18. Method of manufacturing a syringe according to claim 1, the main body (2) is formed by a first co-injection molding shot and the threaded connection part (9) is overmolded to the main body (2) by a second injection molding shot.

19. Method according to claim 17, wherein the first and second molding shots are sequentially carried out in a single mold.
